(19) United States
(12) Patent Application Publication
(10) Pub. No.: US 2022/0061734 A1
(43) Pub. Date: Mar. 3, 2022

(54) METHODS, SYSTEMS AND MEDIA FOR DETRENDING BIOELECTRIC SIGNALS

(71) Applicant: BioSig Technologies, Inc., Westport, CT (US)

(72) Inventors: Alexander David WISSNER-GROSS, Cambridge, MA (US); Suraj Kapa, Rochester, MN (US); James Y. Lee, Stamford, CT (US); Desmond B. Keenan, Stevenson Ranch, CA (US); Natasha Drapeau, Geneva (CH); Kenneth L. Londoner, Westport, CT (US); Julie M. Stephenson, Charlotte, NC (US); Olivier P. Chaudoir, Medway, MA (US)

(21) Appl. No.: 17/411,955

(22) Filed: Aug. 25, 2021

Related U.S. Application Data
(60) Provisional application No. 63/070,093, filed on Aug. 25, 2020.

(51) Int. Cl. A61B 5/367; A61B 5/28; A61B 5/346; A61B 5/00

(52) U.S. Cl. CPC A61B 5/367; A61B 5/28; A61B 2560/02; A61B 5/7225; A61B 5/7271; A61B 5/346

(57) ABSTRACT

Methods, systems, and media are disclosed for detrending a bioelectric signal. In some embodiments, the disclosed system can include a processor configured to receive the bioelectric signal, identify at least one breakpoint section corresponds to a rapid change of amplitude of the bioelectric signal, smooth an amplitude of the bioelectric signal after the at least one breakpoint section; and reconstruct the bioelectric signal based on the smoothed amplitude of the bioelectric signal and a reset of the breakpoint section to remove extrinsic components caused by a non-biological factor from the bioelectric signal.

### CROSS-REFERENCES TO RELATED APPLICATIONS

This application claims the benefit and priority to U.S. Provisional Patent Application No. 63/070,093, filed Aug. 25, 2020, entitled “Methods, Systems And Media For Detrending Bioelectric Signals,” which is incorporated herein by reference in its entirety.

### FIELD

Embodiments included herein generally relate to methods, systems and media for processing bioelectric signals. More particularly, embodiments relate to hardware and software for detrending bioelectric signals.

### BACKGROUND

Electrophysiology is a bioelectrical signal recording technique that enables the measurement and study of electrical properties of biological cells and tissues. For example, cardiac electrophysiology (EP) signals are used by cardiologists to analyze cardiac electrical activity, allowing the cardiologists to measure irregular rhythms during cardiovascular activity and diagnose heart diseases. EP monitoring devices have been developed to facilitate rapid response to life-threatening arrhythmias in patients with acute myocardial infarction, for example. EP monitoring has become widely available in various hospital units, such as intensive care environments, ambulatory telemetry units, operating theatres, and emergency rooms. Multi-electrode EP monitoring, such as electrocardiography, has been developed for detection of complex arrhythmias, identification of prolonged QT intervals, and ST-segment/ischemia monitoring, etc., as understood by those skilled in the art.

However, when an EP measurement procedure (e.g., a cardiac measurement procedure) is performed on a patient, one or more other procedures that involve applying a large amount of energy to one or more biological tissues of the patient may also be performed on the patient simultaneously (e.g., ablation or defibrillation). As such, the EP sensors corresponding to the EP electrodes (e.g., cardiac electrodes) might be temporarily blinded due to the large amount of energy that is being injected into the one or more biological tissues. The temporary blinding of EP sensors makes it impossible for clinicians to get an immediate readout of intrinsic EP signals of interest.

Accordingly, it is desired to provide a system and a method that can detrend the bioelectric signals to remove extrinsic signals from energy injection, such that the clinicians can be de-blinded from the before and aftereffects of injecting large amounts of energy.

### SUMMARY

Methods, systems and media for detrending bioelectric signals are provided.

One aspect of the present disclosure provides a system for detrending a bioelectric signal. The system comprises a processor configured to: receive the bioelectric signal; identify at least one breakpoint section corresponds to a rapid change of amplitude of the bioelectric signal; smooth an amplitude of the bioelectric signal after the at least one breakpoint section; and reconstruct the bioelectric signal based on the smoothed amplitude of the bioelectric signal; reset of the breakpoint section to remove extrinsic components caused by a non-biological factor from the bioelectric signal.

In some embodiments, the system further comprises an electrode configured to collect the bioelectric signal, wherein the bioelectric signal comprises a cardiac electrophysiology signal.

In some embodiments, the processor is further configured to determine a slew threshold indicating a minimum amplitude change for identifying an energy injection event caused by the non-biological factor.

In some embodiments, the processor is further configured to determine if the rapid change of amplitude of the bioelectric signal is larger than the slew threshold and a time point of the rapid change.

In some embodiments, the processor is further configured to identify the at least one breakpoint section based on the determined time point and a parametric temporal radius around the determined time point.

In some embodiments, the processor is further configured to adjust at least one of the slew threshold, the parametric temporal radius, and a smoothing timescale based on a user input.

In some embodiments, the processor is further configured to identify a plurality of breakpoint sections from the bioelectric signal during a period of time. Each of the plurality of breakpoint sections corresponds to one of a plurality of rapid changes of amplitude of the bioelectric signal during the period of time.

In some embodiments, the processor is further configured to: partition the bioelectric signal into a plurality of intervals based on the plurality of breakpoint sections; and independently smooth the amplitude of the bioelectric signal for each interval between adjacent breakpoint sections.

In some embodiments, the processor is further configured to apply a cubic spline smoothing process to smooth the amplitude of the bioelectric signal.

In some embodiments, the processor is further configured to apply a cubic Savitzky-Golay smoothing process to smooth the amplitude of the bioelectric signal.

In some embodiments, the processor is further configured to reset the amplitude of the bioelectric signal in the at least one breakpoint section to a ground baseline.

In some embodiments, the processor is further configured to subtract the smoothed amplitude of the bioelectric signal from the amplitude of the received bioelectric signal.

In some embodiments, the processor is further configured to: collect a plurality of bioelectric signals simultaneously; and simultaneously detrend at least two of the plurality of bioelectric signals in a real time basis.

Another aspect of the present disclosure provides a method for detrending a bioelectric signal. The method comprises: receiving the bioelectric signal; identifying at least one breakpoint section corresponds to a rapid change of amplitude of the bioelectric signal; smoothing an amplitude of the bioelectric signal after the at least one breakpoint section; and reconstructing the bioelectric signal based on the smoothed amplitude of the bioelectric signal and a reset of the breakpoint section to remove extrinsic components caused by a non-biological factor from the bioelectric signal.

In some embodiments, the method further comprises collecting a cardiac electrophysiology signal as the bioelectric signal.

In some embodiments, identifying the at least one breakpoint section comprises determining a slew threshold indicating a minimum amplitude change for identifying an energy injection event caused by the non-biological factor.

In some embodiments, identifying the at least one breakpoint section further comprises determining if the rapid change of the amplitude of the bioelectric signal is larger than the slew threshold and a time point of the rapid change.

In some embodiments, identifying the at least one breakpoint section further comprises identifying the at least one breakpoint section based on the determined time point and a parametric temporal radius around the determined time point.

In some embodiments, the method further comprises adjusting at least one of the slew threshold, the parametric temporal radius, and a smoothing timescale based on a user input.

In some embodiments, the method further comprises identifying a plurality of breakpoint sections from the bioelectric signal during a period of time. Each of the plurality of breakpoint sections corresponds to one of a plurality of rapid changes of amplitude of the bioelectric signal during the period of time.

In some embodiments, the method further comprises: partitioning the bioelectric signal into a plurality of intervals based on the plurality of breakpoint sections; and independently smoothing the amplitude of the bioelectric signal for each interval between adjacent breakpoint sections.

In some embodiments, smoothing the amplitude of the bioelectric signal comprises applying a cubic spline smoothing process to smooth the amplitude of the bioelectric signal.

In some embodiments, smoothing the amplitude of the bioelectric signal comprises applying a cubic Savitzky-Golay smoothing process to smooth the amplitude of the bioelectric signal.

In some embodiments, reconstructing the bioelectric signal comprises resetting the amplitude of the bioelectric signal in the at least one breakpoint section to a ground baseline.

In some embodiments, reconstructing the bioelectric signal further comprises subtracting the smoothed amplitude of the bioelectric signal from the original amplitude of the bioelectric signal.

In some embodiments, the method further comprises: collecting a plurality of bioelectric signals simultaneously; and simultaneously detrending at least two of the plurality of bioelectric signals in a real time basis.

Another aspect of the present disclosure provides a non-transitory computer-readable medium containing computer-executable instructions that, when executed by a hardware processor, cause the hardware processor to perform the disclosed method for detrending bioelectric signals.

Other aspects of the present disclosure can be understood by those skilled in the art in light of the description, the claims, and the drawings of the present disclosure.

### BRIEF DESCRIPTION OF THE DRAWINGS

The accompanying drawings, which are incorporated herein and form part of the specification, illustrate the present embodiments and, together with the description, further serve to explain the principles of the present embodiments and to enable a person skilled in the relevant art(s) to make and use the present embodiments.

FIG. 1 illustrates a block diagram of a hardware system including an EP workstation and an EP console, in accordance with some embodiments of the disclosed subject matter.

FIG. 2 illustrates a flowchart of an example method for detrending a bioelectric signal, in accordance with some embodiments of the disclosed subject matter.

FIG. 3 illustrates a schematic diagram of an example interface including a plurality of bioelectric signals before and after an energy injection event, in accordance with some embodiments.

FIG. 4A illustrates a schematic diagram of an example bioelectric signal before and after an energy injection event, in accordance with some embodiments.

FIGS. 4B-4E illustrate enlarged views of example sub-portions of the bioelectric signal shown in FIG. 4A, in accordance with some embodiments.

FIG. 5A illustrates a schematic diagram of identifying a plurality of breakpoint sections of the example bioelectric signal shown in FIG. 4A, in accordance with some embodiments.

FIGS. 5B-5E illustrate enlarged views of example sub-portions of the bioelectric signal shown in FIG. 5A, in accordance with some embodiments.

FIG. 6A illustrates a schematic diagram of the example bioelectric signal including smoothed intervals, in accordance with some embodiments.

FIGS. 6B-6E illustrate enlarged views of example sub-portions of the bioelectric signal shown in FIG. 6A, in accordance with some embodiments.

FIGS. 7A, 7F and 7G illustrate schematic diagrams of example detrended bioelectric signals, in accordance with some embodiments.

FIGS. 7B-7E illustrate enlarged views of example sub-portions of the detrended bioelectric signal shown in FIG. 7A, in accordance with some embodiments.

FIGS. 8A-8E illustrate schematic diagrams of example interfaces including a plurality of bioelectric signals after a detrending process, in accordance with some embodiments.

The features and advantages of the present embodiments will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION

Although specific configurations and arrangements are discussed, it should be understood that this is done for illustrative purposes only. A person skilled in the pertinent art will recognize that other configurations and arrangements can be used without departing from the spirit and scope of the present disclosure. It will be apparent to a person skilled in the pertinent art that the present disclosure can also be employed in a variety of other applications.

It is noted that references in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases do not necessarily refer to the same embodiment. Further, when a particular feature, structure or characteristic is described in connection with an embodiment, it would be within the knowledge of a person skilled in the pertinent art to effect such feature, structure or characteristic in connection with other embodiments whether or not explicitly described.

In general, terminology may be understood at least in part from usage in context. For example, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context.

Methods, systems, and media are disclosed for detrending bioelectric signals to remove extrinsic components caused by a non-biosocial factor.

This disclosure refers to electrophysiology (EP) signals (e.g., cardiac EP signals, electroneuronographic signals, etc.) taken from potential differences recorded at two (or more) different, separated electrodes that are placed on or in a patient's body. For example, the EP signals can include SECG signals measured from surface leads placed on limbs and chest of the patient. As another example, the EP signals can include IECG signals measured from intracardiac (IC) leads, which may include one or more separate catheters placed directly on cardiac tissue of the patient. As yet another example, the EP signals can include electroneuronographic (ENOG) signals such as deep brain stimulation (DBS) signals from DBS leads on a lead wire implanted into a specific brain area of the patient, spinal cord stimulation (SCS) signals from SCS leads placed under the skin of the patient, and/or sacral neuromodulation (SNM) signals from SNM leads on a lead wire implanted near the sacral nerve of the patient, etc.

As described in the background section, the clinicians can be temporarily blinded because the EP signals can be disturbed due to the before and after effect of injecting a large amount of energy into a biological tissue (e.g., the cardiac tissue) of a patient. For example, during an ablation procedure, a large amount of energy can be injected into a patient in order to burn a biological tissue, such that the biological tissue can be deranged in order to restore periodic for resolving an arrhythmia. As another example, during a pacing procedure, an artificial heartbeat can be created in order to give a biological tissue a hint as to the phase and periodicity with which action potentials should be firing. As yet another example, during a cardioversion procedure, the entire patient can be electrically shocked. In various scenarios that a large amount of electrical energy is injected into a biological tissue (e.g., the cardiac tissue) with good intentions, the EP signals can be disturbed, thereby resulting a temporary blinding of clinicians to the intrinsic signals of interest.

Further, in some situations, the clinicians may use unipolar sensors instead of bipolar sensors in the EP signal monitoring devices. In such situations, the potential differences relative to a floating ground potential can be used instead of using potential differences between two closely spaced electrodes. Due to the floating ground, the baseline potential of an EP sensor may slowly drift, resulting in a low frequency noise cancellation problem of the EP sensory signals, thereby reducing the ability of clinicians to identify the intrinsic signals.

In order to resolve the at least two issues described above, the disclosed methods, systems, and media can detrend the EP signals to remove extrinsic signals resulting from the energy being injected into a biological tissue, and to remove artifact components due to the floating round type problems (e.g., drifting baseline potentials) caused by using unipolar sensors as opposed to bipolar sensors.

This disclosure identifies both hardware and software embodiments to achieve these objectives.

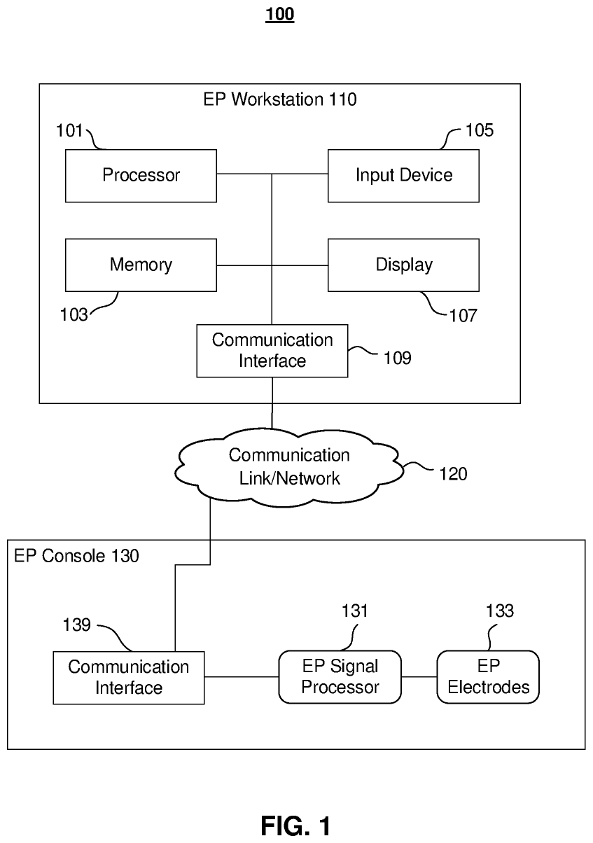

FIG. 1 is a block diagram representing an example hardware system 100, including, for example, an EP workstation 110 and an EP console 130 that can be connected via a communication link/network 120, in accordance with some embodiments of the disclosed subject matter.

As shown in FIG. 1, EP workstation 110 can include user input, visualization, and review functionalities. For example, EP workstation 110 can include one or more processors 101, memory 103, one or more local and/or remote displays 107, one or more input devices 105, and a communication interface 109, which can be interconnected via a communication infrastructure or bus (not shown).

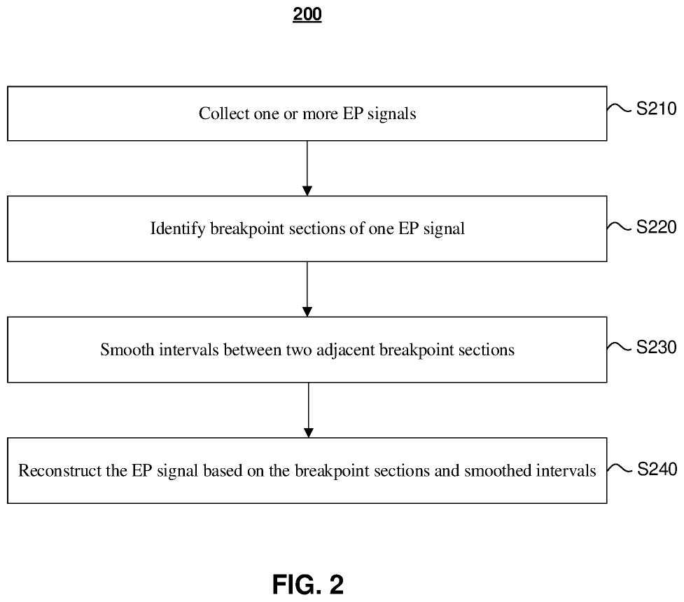

One or more processors 101 can use a computer program to execute the mechanisms described herein, including performing the method for detrending EP signals as described below in connection with FIG. 2; sending and/or receiving data through communications interface 109; sending and/or receiving raw EP signals transmitted by an EP console 130; and/or performing any other suitable actions. In some embodiments, one or more processors 101 can send and receive data through communications interface 109 or any other communication links using, for example, a transmitter, a receiver, a transmitter/receiver, a transceiver, or any other suitable communication device.

In some embodiments, memory 103 can include a storage device (such as a computer-readable medium) for storing a computer program for controlling processor 101. In some embodiments, one or more local and/or remote displays 107 can include a touchscreen, a flat panel display, a projector, a speaker, and/or any other suitable display and/or presentation devices for providing display capability for EP signal visualization and review software. One or more input devices 105 can include, for example, a computer keyboard, a mouse, a keypad, a remote control, a microphone, a touchscreen, and/or any other suitable input device as would be used by a designer of input systems or process control systems.

EP console 130 can include multiple EP electrodes 133 configured to be placed on various locations on or in a patient's body for collecting or measuring electrophysiology (EP) signals (e.g., cardiac EP signals, electroneuronographic signals, etc.) of the patient. For example, the multiple EP electrodes 133 can include a plurality of surface electrodes placed on a patient's skin, and/or a plurality of intracardiac (IC) electrodes placed inside the patient's heart. EP console 130 can include one or more EP signal processors 131 to process the EP signals from the multiple EP electrodes 133. In some embodiments, raw EP signals may be small analog signals, which may require conditioning and amplification to be accurately evaluated and transformed to digital EP signals.

It is noted that, EP console 130 can be used for measuring any suitable electroneuronographic (ENOG) signals. In some embodiments, EP console 130 can be a cardiac signal detecting system used for collecting cardiac EP signals. In some other embodiments, EP console 130 can include any other suitable ENOG signal detecting systems, such as a deep brain stimulation (DBS) signal detecting system, a spinal cord stimulation (SCS) signal detecting system, a sacral neuromodulation (SNM) signal detecting system, etc. In some embodiments, EP console 130 can be an integrated system including any suitable combination of various subsystems for detecting various EP signals, respectively.

Communications network 120 can be any suitable wired or wireless (or a combination thereof) computer network or combination of networks including the Internet, an intranet, a wide-area network (“WAN”), a local-area network (“LAN”), a wireless network, a digital subscriber line (“DSL”) network, a frame relay network, an asynchronous transfer mode (“ATM”) network, a virtual private network (“VPN”), etc.

Communication interfaces 109 and 139 can be any interfaces suitable for communicating data between EP workstation 110 and EP console 130. Communication interfaces 109 and 139 can enable EP workstation 110 and EP console 130 to communicate and interact with any combination of external devices, external networks, external entities, etc. EP workstation 110 and EP console 130 can be located in a same location or remotely separated.

Referring to FIG. 2, a flowchart of an example method 200 for detrending EP signals is shown in accordance with some embodiments of the disclosed subject matter.

Method 200 can start at operation 5210, in which one or more EP signals can be collected. In some embodiments, the one or more EP signals can be directly collected by an EP console, as discussed above in connection with FIG. 1. For example, row analog EP signals can be detected by a plurality of EP electrodes. The row analog EP signals can then be processed by one or more EP signal processors. For example, the row analog EP signals can be amplified by one or more EP signal amplifiers, and then converted to digital EP signals by an analog-to-digital converter.

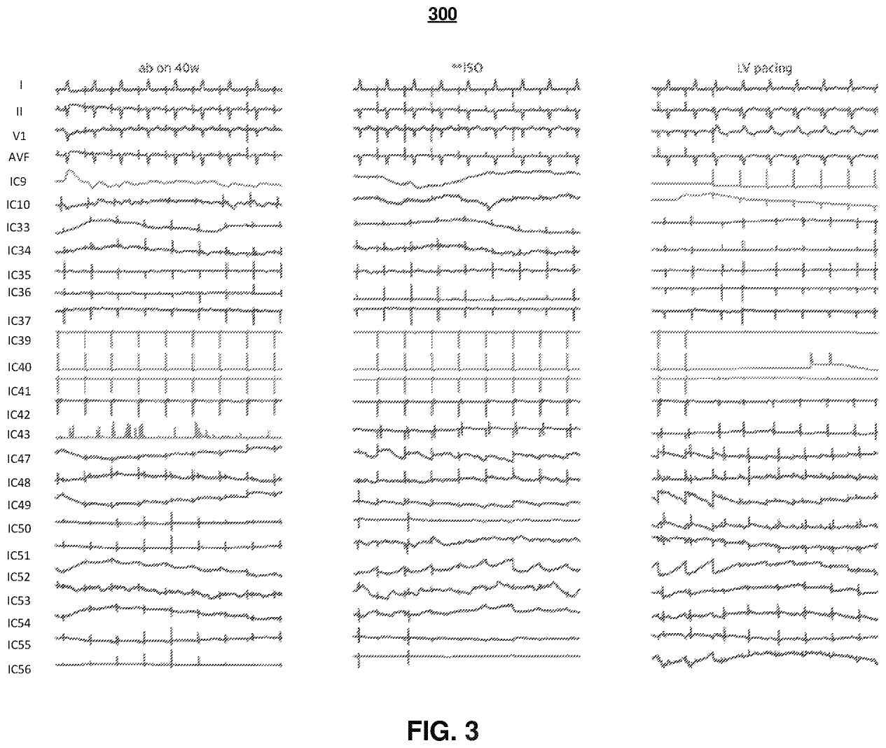

In some embodiments, multiple EP signals can be collected and processed simultaneously in a real time basis, and can be presented in any suitable manner. For example, as shown in FIG. 3, a plurality of EP signals can be collected from a plurality of cardiac EP electrodes, and can be presented in parallel in a real time basis in a user interface 300 by display 107 of EP workstation 110.

It is noted that cardiac EP signals (e.g., electrocardiography (ECG) signals) are used as an example and a representative for the EP signals in the following description of method 200 for demonstrative propose, but this should not limit the disclosed subject matter. Method 200 can be applied to any other suitable EP signals, including, but not limited to electroneuronographic (ENOG) signals, electroencephalographic (EEG) signals, electromyographic (EMG) signals, electrooculography (EOG) signals, electrocochleographic (ECOG) signals, electrogastrographic (EGG) signals, electrogastroenterographic (EGEG) signals, electrohysterographic (EHG) signals, electropneumographic (EPG) signals, electrospinographic (ESG) signals, etc.

As described above, in some embodiments, when the cardiac procedures are performed, and a broad class of other procedures are also performed such that a large amount of energy may be injected into one or more biological tissues. Thereby, one or more EP electrodes placed close to the one or more biological tissues may be temporarily blinded due to the injected energy. As shown in FIG. 3, the EP signals of the electrodes IC9, IC39, IC40, IC41, and IC43 are disturbed by the injected energy of an ablation (isolation) event, thus the presented waveforms of the corresponding EP signals have abnormal shapes.

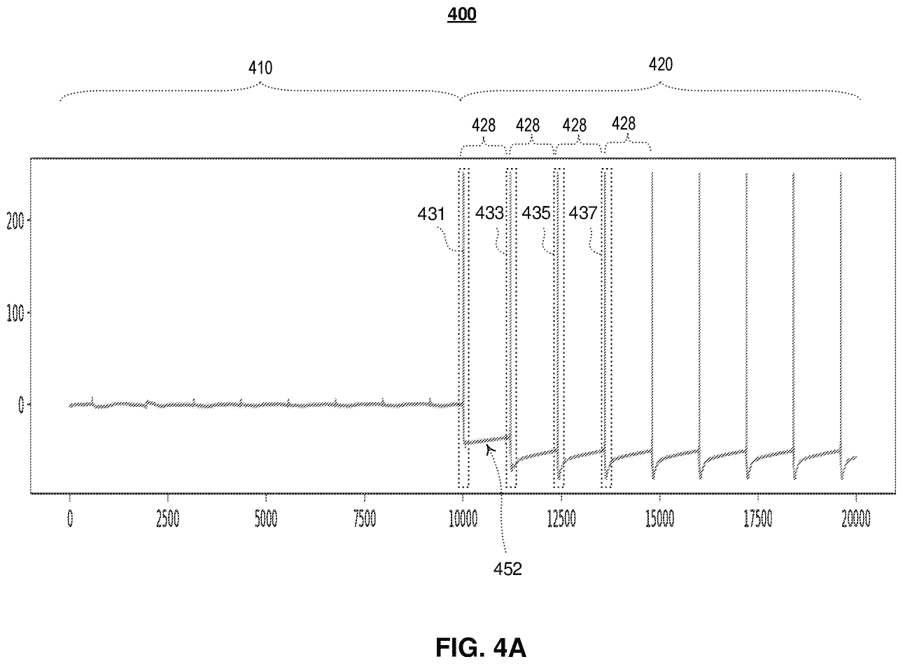

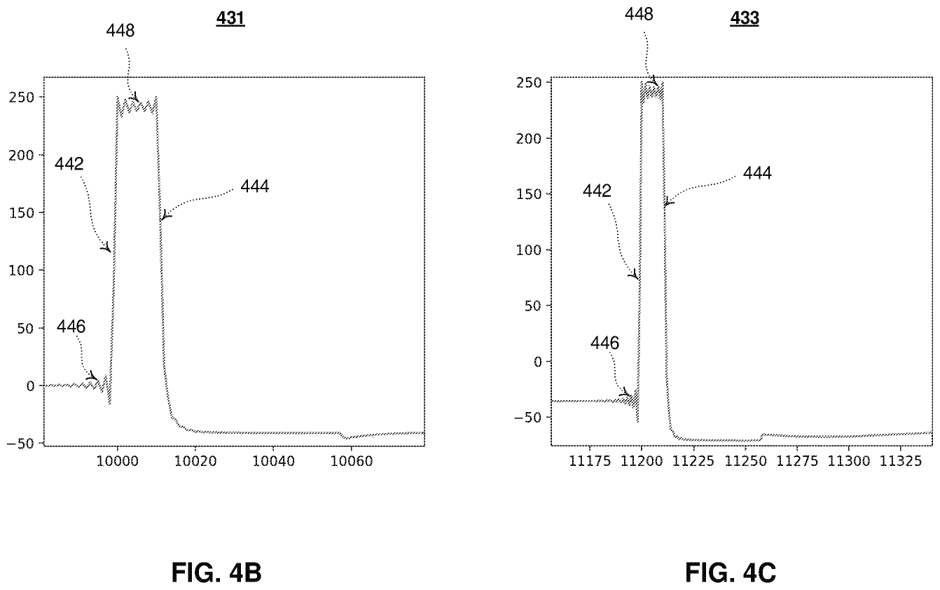

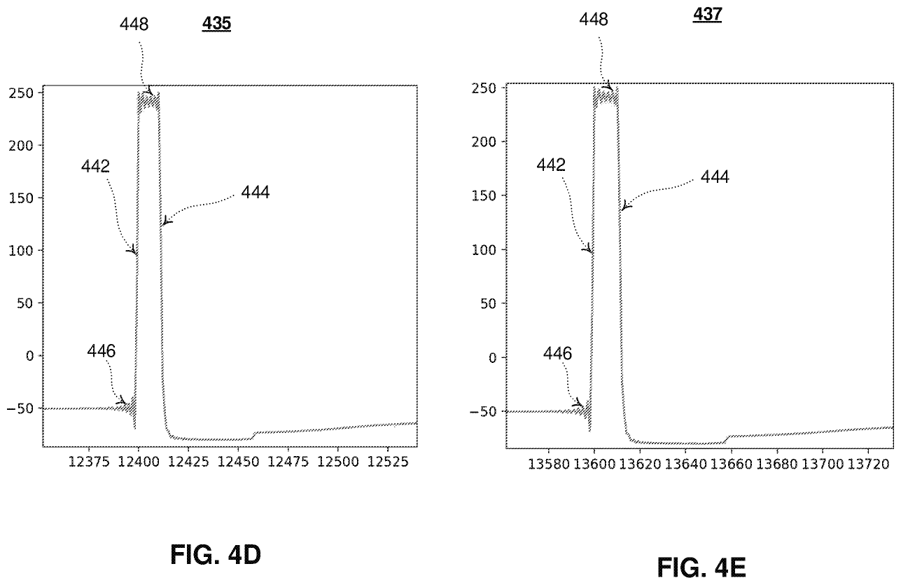

Referring to FIG. 4A, waveforms of an EP signal before and after injecting a large amount of energy are shown in accordance with some embodiments. The EP signal is collected from ABL-d channel (IC9 electrode) before and after an ablation (isolation) event, and is used herein as an example. It is noted that, the disclosed method can be applied to multiple EP signals collected from multiple EP electrodes simultaneously in a parallel real time basis. It is also noted that, the disclosed method can be also applied to any other events that involve a large amount of energy injected in, such as a cardioversion procedure, a stereotaxic procedure, etc.

The left portion is the normal waveforms 410 of the EP signal that has not been affected by the energy disturbing of the ablation event, while the right portion is the abnormal waveforms 420 of the EP signal that has been effected by the energy disturbing of the ablation event.

In the normal waveforms 410 of the EP signal, expect the deflections, the potentials associated with intrinsic signals is on an order of a few millivolts. In some embodiments, the peak to trough voltages of the EP signal is generally less than few dozens of millivolts (e.g., six or seven millivolts).

However, the injected large amounts of energy (e.g., in the ablation event) can have a high voltage, such as hundreds of millivolts (e.g., 250 millivolts), which can easily saturate the voltage limits of the EP sensor system. As shown in the right portion of the abnormal waveforms 420 of the EP signal, the injected energy can introduce high-voltage peaks in each interval 428 of the abnormal waveforms of the EP signal, which significantly change the horizontal aspect ratio of the EP signal waveforms, and causes the biological information of interest carried by the normal waveforms with the low-voltage portions of the EP signal being obscured in the noises of the extrinsic signals.

FIGS. 4B-4E illustrate enlarged views of the portions 431, 433, 435, and 437 of the abnormal waveforms 420 of the EP signal as shown in FIG. 4A. Is it noted that, in the following description, various potions of only four intervals 428 are illustrated as examples for illustrative purpose and simplicity, but not limit the scope of the present disclosure. The disclosed method can be applied to a plurality of intervals 428, where the number of the plurality of intervals 428 is not limited.

Referring to FIGS. 4B-4E, each of the high slope segments 442, 444 in each interval 428 indicates a huge voltage jump over a very short period of time that corresponds to an energy injection event (e.g., an ablation event). Such voltage change is too fast to be caused by a biology factor, thus is also referred as an artifact of slow filtering. The EP signal is sampled at a finite rate (e.g., two kilohertz), an instantaneous voltage change can be represented effectively by using a step function. The high slope segments 442, 444 on the boundaries of a series of intervals 428 represent a chain of high-voltage pulses over biological improbable periods of time, which are the first type of artifacts or extrinsic signals that are to be removed in the subsequent processes.

The vibration segments 446, 448 are the second type of artifacts, which indicate the settling time before and after the large potential change event. When the corresponding EP signal is processed by the EP signal processor 131, the step function representing an instantaneous voltage change is processed through a low pass filter, a settling behavior including a ringing with an exponential decay both before and after the step function is illustrated as the vibration segments 446, 448. Such second artifacts are not caused by a biology factor but caused by physics and signal processing. Therefore, such second type of artifacts are also non-intrinsic signals and are to be identified and removed in the subsequent processes.

The ascending segments 452 as shown in FIG. 4A are the third type of artifacts due to one or more non-biological factors, such as residual parasitic capacitance. The intrinsic signals are dropped from a biological baseline for sinus rhythm closing to the ground voltage (e.g., zero baseline waveform 410 in FIG. 4A) to a range from about negative 80 millivolts to about negative 40 millivolts, and are zoomed out due to the scaling of the vertical axis. As such, the resulted scaled version of the abnormal waveforms 420 may cause the clinicians to lose the ability to see any features of interest of the intrinsic signals on the vertical axis. Therefore, the third type of artifacts are also extrinsic signals, and are to be removed in the subsequent processes to expose the features of interest of intrinsic signals.

Referring back to FIG. 2, method 200 can proceed to operation S220, in which at least one breakpoint section of at least one EP signal can be identified. In some embodiments, during breakpoint section identification, a time section around a quick change of the at least one EP signal that is not caused by a biology factor but caused by some extrinsic factors can be identified as a breakpoint section.

In some embodiments, the breakpoint section identification can include determining a slew threshold that indicates a minimum change in volts per second for identifying an energy injection event caused by a non-biological factor. The predetermined slew threshold can be used for triggering an identification of a breakpoint section due to the voltage change being too rapid to have been biological in origin. Specifically, the real time EP signal can be sampled in a proper rate to identify the absolute values of the voltage change between consecutive samples. The absolute values of the voltage change between consecutive samples can be filtered by comparing to the slew threshold. If one absolute value of the voltage change between a pair of consecutive samples is larger the slew threshold, the time points corresponding to the high slope segments 442, 444 as shown in FIGS. 4B-4E can be determined. It is noted that, the slew threshold can be either predetermined or tunable. In some embodiments, a value of the slew threshold can be set within a range from 1,000 mV/s to 10,000 mV/s, such as about 5,000 mV/s.

In some embodiments, the breakpoint section identification can further include setting a parametric temporal radius around the determined time points to solve the settling issue. The parametric temporal radius can be either predetermined or tunable. In some embodiments, a value of the parametric temporal radius can be not less than a maximum width of the plurality of vibration segments 448 shown in FIGS. 4B-4E. For example, the parametric temporal radius can be set within a range from 10 ms to 30 ms, such as about 20 ms. Since the parametric temporal radius is set around those time points where there are rapid voltage changes, the voltage within the parametric temporal radius before and after the identified time points can be zeroed. That is, any voltage changes of the sampled EP signal that happened within a certain time period before and after a slew threshold change in voltage can be removed. As such, the second type of artifacts described above corresponding to the vibration segments 446, 448 shown in FIGS. 4B-4E due to the settling issue can be removed.

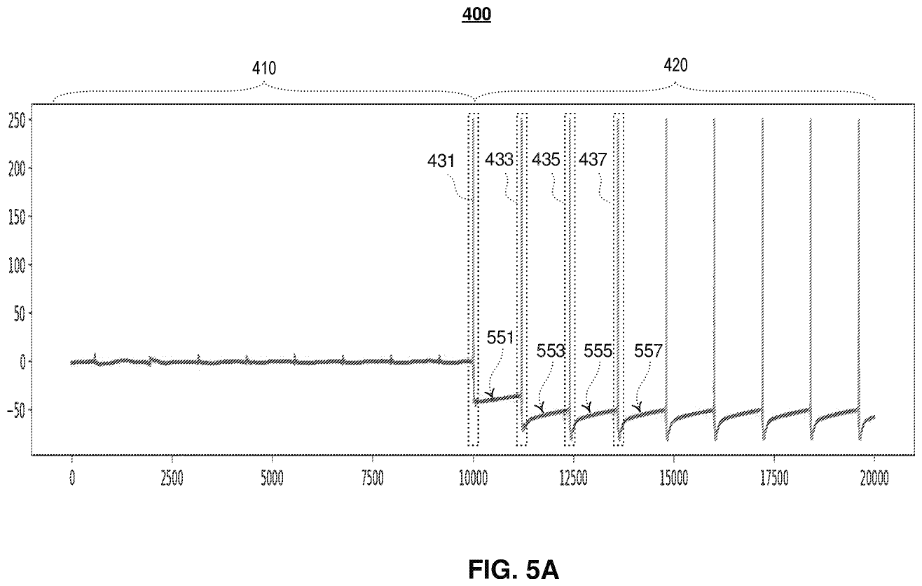

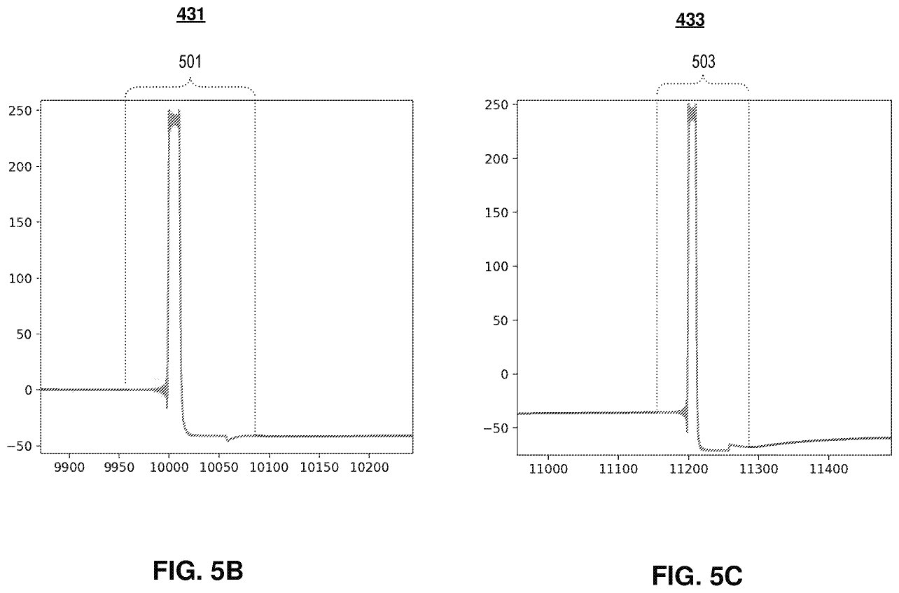

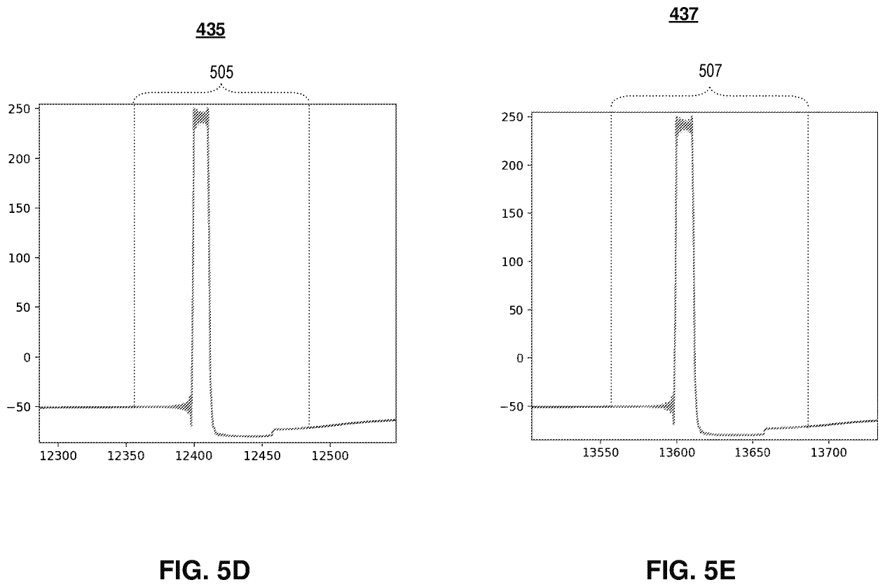

Accordingly, based on the above two steps, a plurality of breakpoint sections 501, 503, 505, 507, etc., of the EP signal can be identified over the time, as shown in FIGS. 5A-5E, while the voltage changes in vibration segments 446, 448 have been zeroed.

In some embodiments, based on the identified breakpoint sections (e.g., breakpoint sections 501, 503, 505, 507, etc., as shown in FIGS. 5B-5E) of the EP signal, the abnormal waveforms 420 of the EP signal can be partitioned up into a set of intervals 551, 553, 555, 557, etc., as shown in FIG. 5A. Each interval can be defined as the EP signal segment between two adjacent identified breakpoint sections.

Referring back to FIG. 2, method 200 can proceed to operation S230, in which at least one interval between two adjacent identified breakpoint sections can be smoothed. In some embodiments, the smoothing can be designed to isolate the lower frequency components. As such, the slowly changing trend line of each interval can be extracted out the to identify baseline wandering, parasitic capacitance, or any other non-biology factors. Any suitable smoothing method, such as spline smoothing (e.g., cubic or higher order spline smoothing), Savitzky-Golay smoothing (e.g., cubic or higher order S-G smoothing), etc., can be used in operation S230 to independently smooth each interval sequentially over the time.

In some embodiments, cubic spline smoothing can be used as a piecewise regression technique to do regression for pieces of a function (e.g., each interval partitioned in operation S220). For example, given a piecewise function, a cubic function can be fitted to each discrete piece of the piece function with the boundary conditions such that the cubic functions line-up with the boundaries between the pieces to satisfy a desired smoothness condition. That it, cubic splines impose that both the cubic functions and the boundaries between the piecewise cubic functions are continuously differentiable. In some embodiments, the connections of the cubic splines can be desirably smooth, so that the boundaries between the pieces (also referred as “knots”) are not recognizable, a smooth fit to the slowly changing waveforms of each interval of the EP signal can be independently obtained. In some embodiments, a smoothing timescale of the spline smoothing can be tunable. In some embodiments, a smoothing timescale of the spline smoothing can be set as around the temporal distance between spline knots, e.g., every 100 samples between spline knots. It is noted that, the spline smoothing can work reasonably well for a slow voltage change. However, if there is an intrinsic change on a certain timescale that is too close with the timescale of the knots between the cubic pieces, the result can include ringing effects due to harmonic interactions between the knots and the intrinsic signal.

In some alternative embodiments, Savitzky-Golay smoothing (“S-G smoothing” hereinafter) can be employed instead of spline smoothing. S-G smoothing can be similar to a moving average process which is a special case of a statistical smoothing. The difference from the moving average process is that the S-G smoothing can include a moving in a non-zero order fit. In a moving average process, a moving window can be defined, and an average value of the function within the moving window can be calculated. Based on the average of a moving window, zero-order polynomials (i.e., constants) can be fitted to the values within the moving window. In the S-G smoothing, instead of fitting a moving average, a moving linear fit (first order polynomials), a moving quadratic fit (second order polynomials), a moving cubic fit (third order polynomials), or arbitrarily high order polynomials, can be applied to each moving window. In some embodiments, a third order polynomial (cubic) fitting of S-G smoothing can have the ability to make a desired smoothing to the abnormal waveforms 420 of the EP signal to remove the artifacts due to the placement of knots.

Because the polynomial fit involved a moving window, care must be taken when defining the size of the moving window. In some embodiments, the size of the moving window can be within an exemplary range from about 60 samples to about 140 samples. In some embodiments, the smoothing timescale of the S-G smoothing can be tunable. In one example, the smoothing timescale of the S-G smoothing can be in a range from 12.5 ms to 500 ms, which can be the same order of magnitude as a sinus rhythm.

It is noted that, there is a desire for the clinicians to have as real time visibility into the EP signal coming out of the patient as possible. However, in the signal processing, it is convenient to smooth the signal after the fact. Thus, in some embodiments, a time deferred smoothing can be used with the timescale of the moving window being set as a halfway region between the timescales of causal smoothing and acausal smoothing. By using the time deferred smoothing, the processed EP signal is not presented to the clinicians in a perfect real time. There is a buffer time up to multi milliseconds to allow the system to do acausal smoothing. Comparing to the zero-delay high order causal smoothing with a plurality of artifacts in the resulted signal, the cubic S-G smoothing with a multi milliseconds delay can provide the clinicians a cleaner, more recognizable real time signal with an acceptable or negligible time lag.

Is it also noted that, in some embodiments, the S-G smoothing process can be triggered by the identification of a breakpoint section as described above. That is, the S-G smoothing process can be operated to the intervals in the abnormal waveforms 420 of the EP signal as shown in FIG. 5A. In some other embodiments, without being triggered by the identification of a breakpoint section, the S-G smoothing can also be operated to the entire EP signal including both normal waveforms 410 and abnormal waveforms 420 of the EP signal.

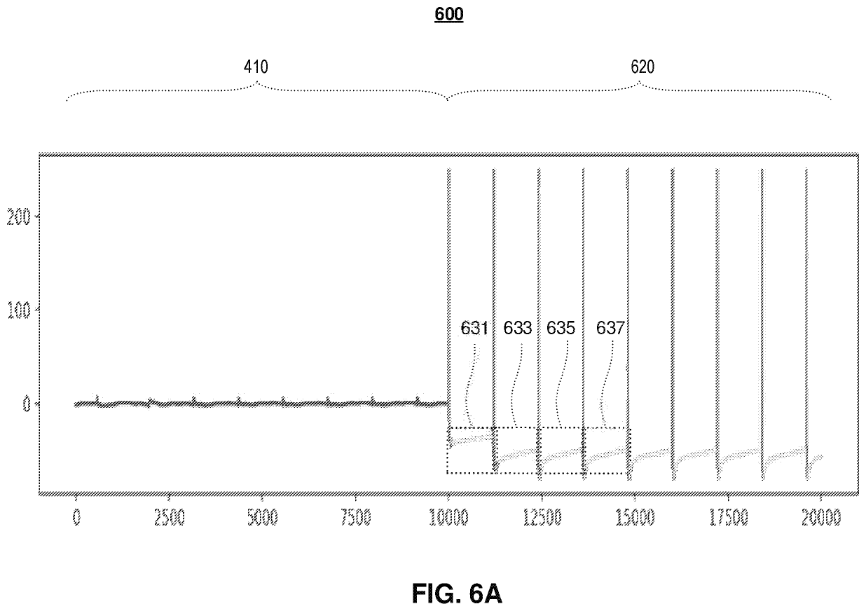

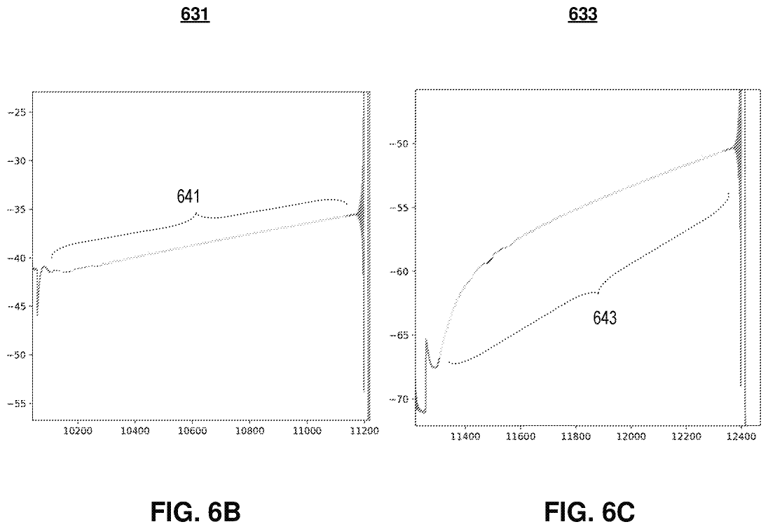

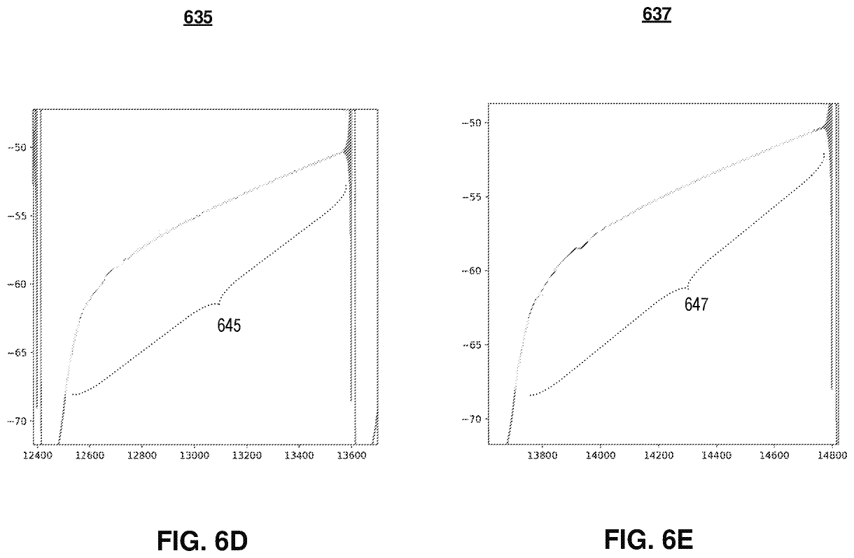

Referring to FIG. 6A, waveforms of the EP signal after a smoothing process are shown in accordance with some embodiments. The smoothing process (e.g., a cubic spline smoothing, a cubic S-G smoothing) is applied to each interval of the abnormal waveforms. FIGS. 6B-6E illustrate enlarged views of the portions 631, 633, 635, 637 of the resulted waveforms 620 of the EP signal after the smoothing process, including the smoothed intervals 641, 643, 645, and 647.

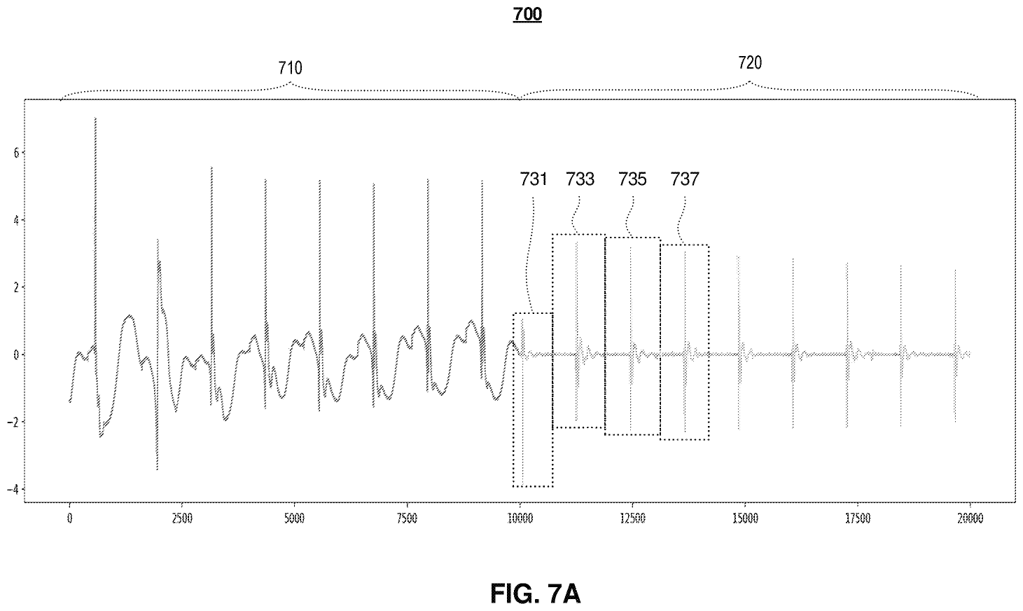

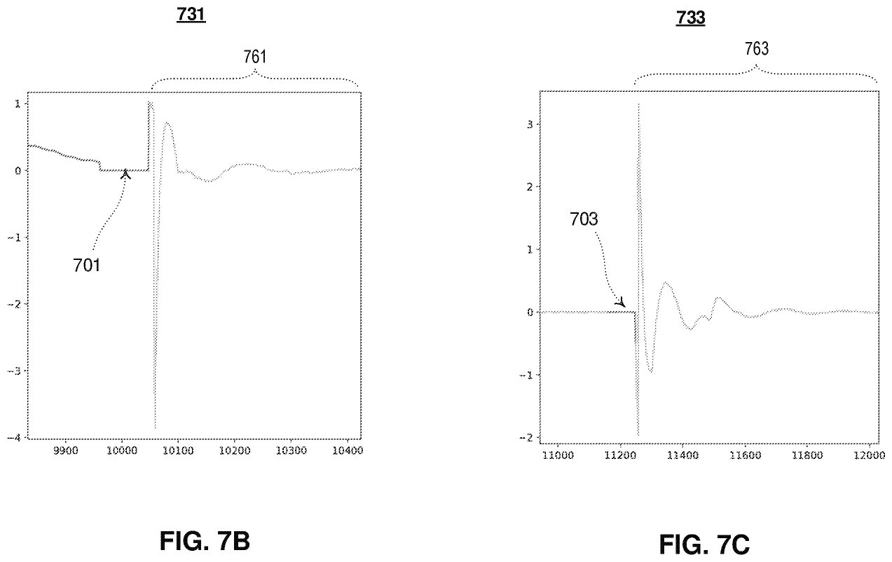

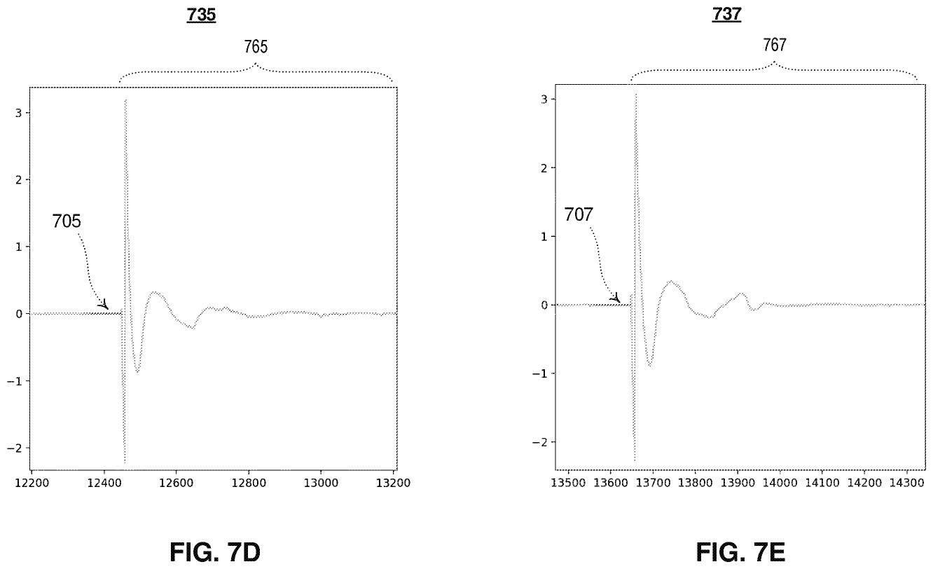

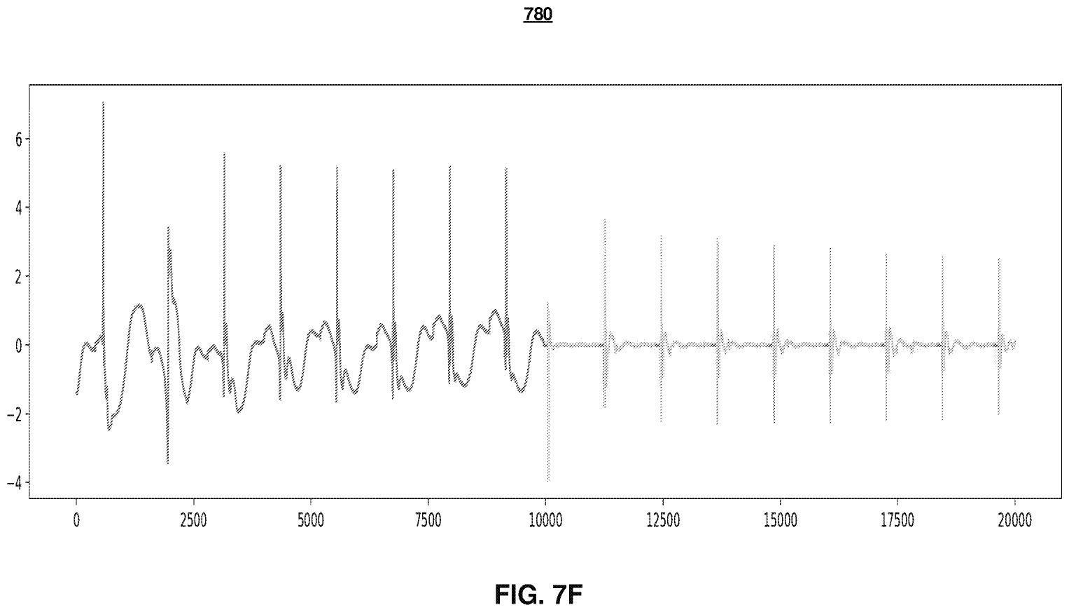

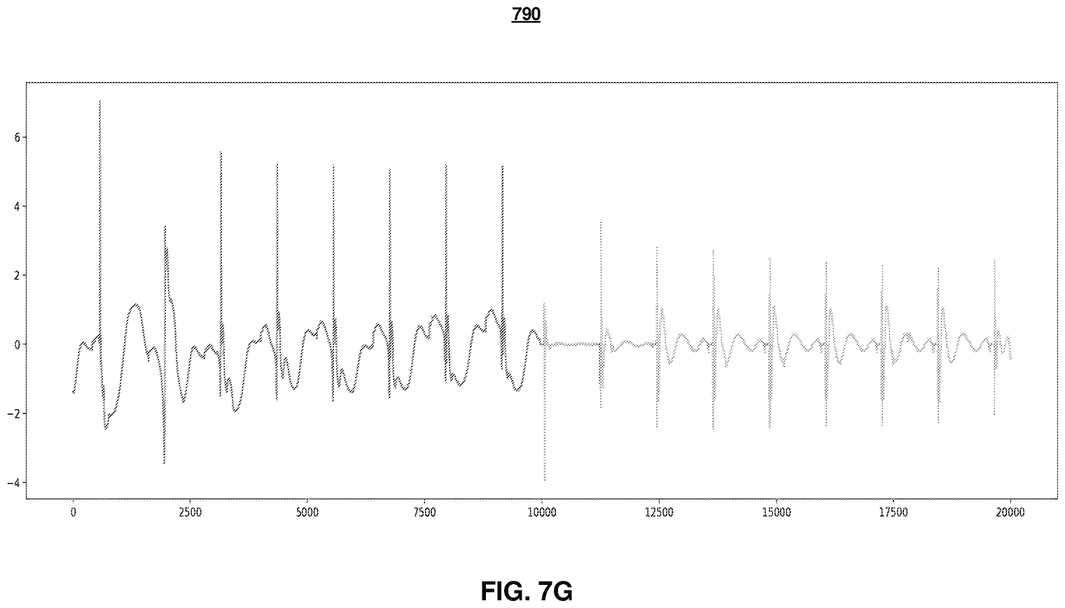

Referring back to FIG. 2, method 200 can proceed to operation S240, in which the at least one EP signal can be reconstructed based on the at least one breakpoint section and the at least one smoothed interval. Referring to FIG. 7A, waveforms 700 of the EP signal after a reconstruction process are shown in accordance with some embodiments. FIGS. 7B-7E illustrate enlarged views of the portions 731, 733, 735, 737 of the reconstructed waveforms of the EP signal.

In some embodiments, the reconstruction process can include resetting the voltage value in the at least one breakpoint section to ground voltage (e.g., zero). That is, the buffers 701, 703, 705, 707, etc., between the intervals are re-set to zero, as shown in FIGS. 7B-7E. It is noted that, the operation of resetting the voltage value in the breakpoint section to ground voltage can be performed simultaneously with the operation S220 of identifying the at least one breakpoint section described above.

In some embodiments, the reconstruction process can further include subtracting the at least one smoothed interval from the EP signal. As such, the features of interest can be recovered for each interval. As shown in FIGS. 7A-7E, the detrended intervals 761, 763, 765, 767, etc., each include recovered intrinsic waveforms of the EP signal. It is noted that, the operation of include subtracting the at least one smoothed interval from the EP signal can be performed simultaneously with the operation S230 of smoothing the at least one interval described above.

It is noted that, FIGS. 7A-7E illustrate the detrended EP signal based on a cubic S-G smoothing with a size of the moving window of a number of 101 samples of each interval. As described above, the size of the moving window of the S-G smoothing is adjustable, which means the smoothing timescale is adjusted accordingly. FIG. 7F illustrates a detrended EP signal 780 based on a cubic S-G smoothing with a size of the moving window of a number of 201 samples of each interval. FIG. 7G illustrates a detrended EP signal 790 based on a cubic S-G smoothing with a size of the moving window of 401 samples of each interval. Comparing to FIGS. 7A, 7F and 7G, it can be found that when the S-G smoothing has a higher sample rate, which indicates a smaller smoothing timescale, can be used to recover more details of the intrinsic waveforms of the EP signal.

It is noted that, comparing to the cubic spline detrending process (referring back the operation S230), the cubic S-G detrending process can have many advantages, such as revealing additional out-of-phase pulses, revealing 60-Hz electric hum in background, avoiding spline knot oscillations, preserving “far-field” intrinsic signals (with >=500-ms windows).

It is also noted that method 200 can be performed by processing logic that can comprise hardware (e.g., circuitry, dedicated logic, programmable logic, microcode, etc.), software (e.g., instructions executing on a processing device), or a combination thereof. It is to be appreciated that not all operations may be needed to perform the disclosure provided herein. Further, some of the steps may be performed simultaneously, or in a different order than shown in FIG. 2, as will be understood by a person of ordinary skill in the art.

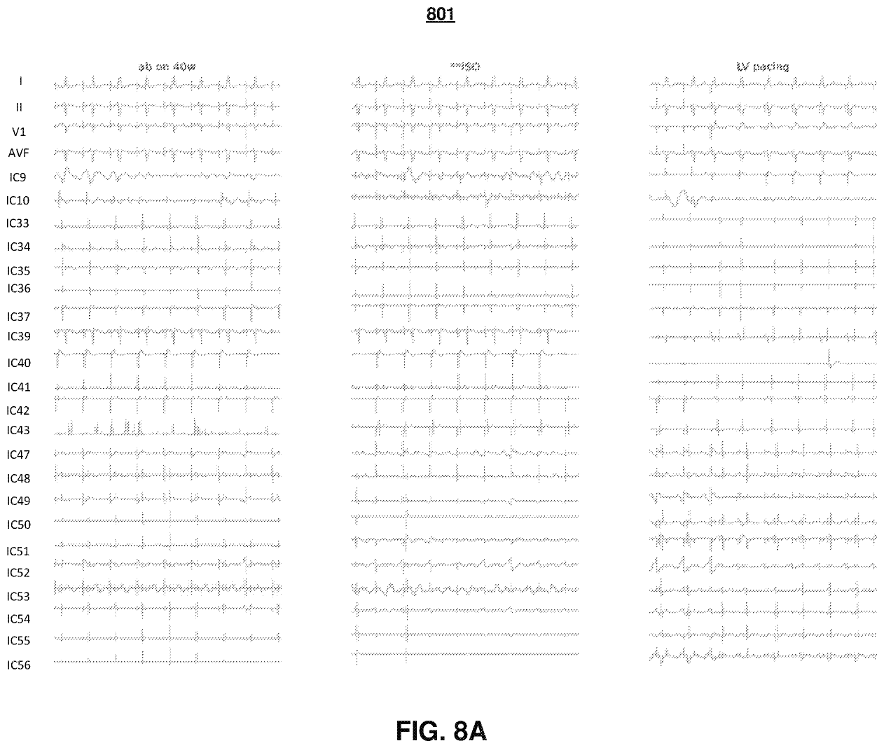

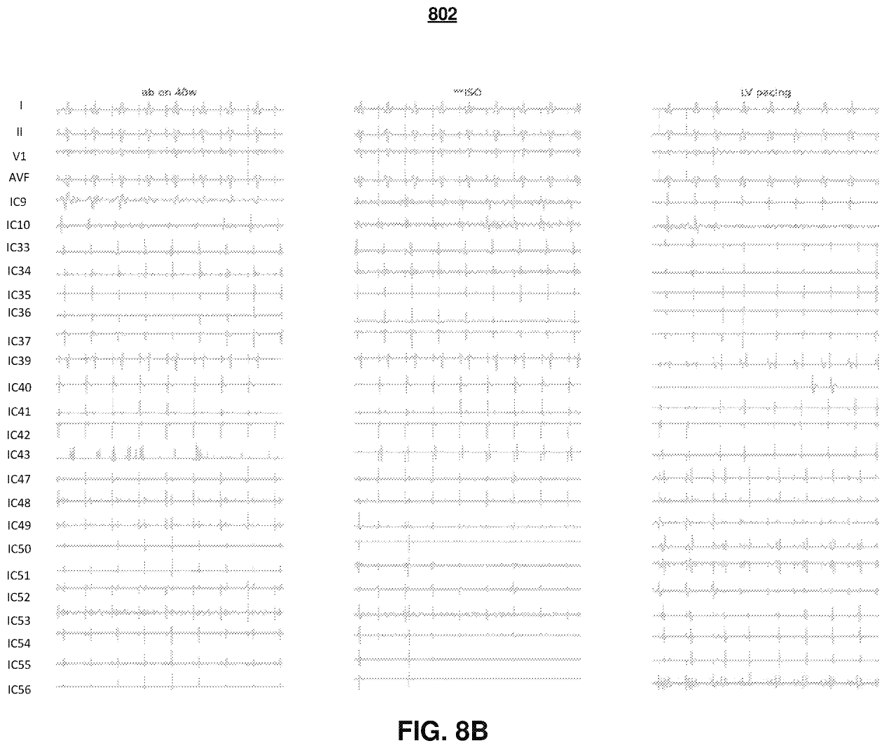

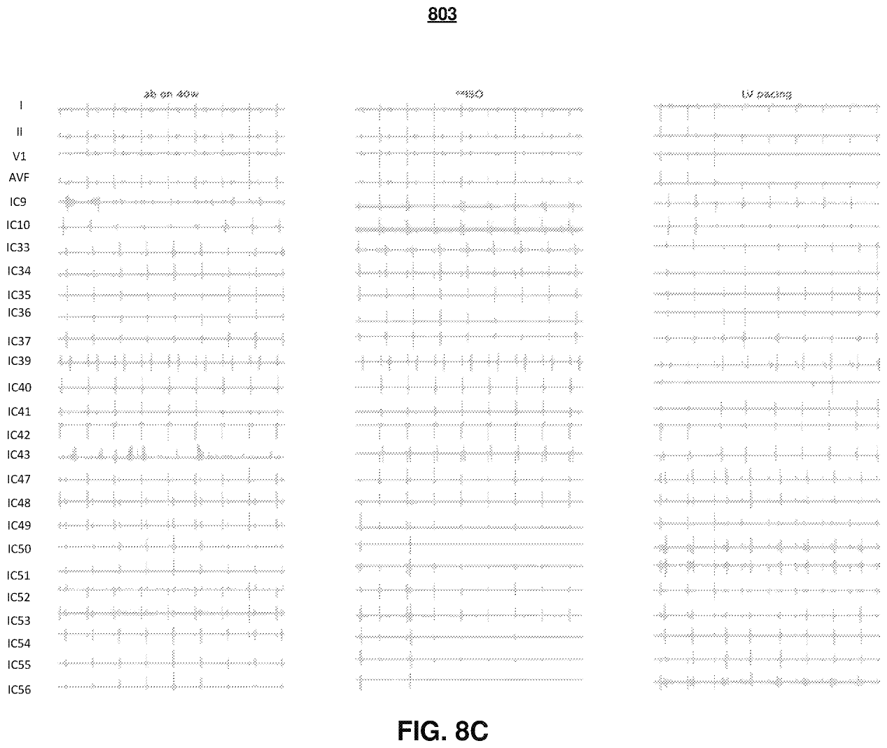

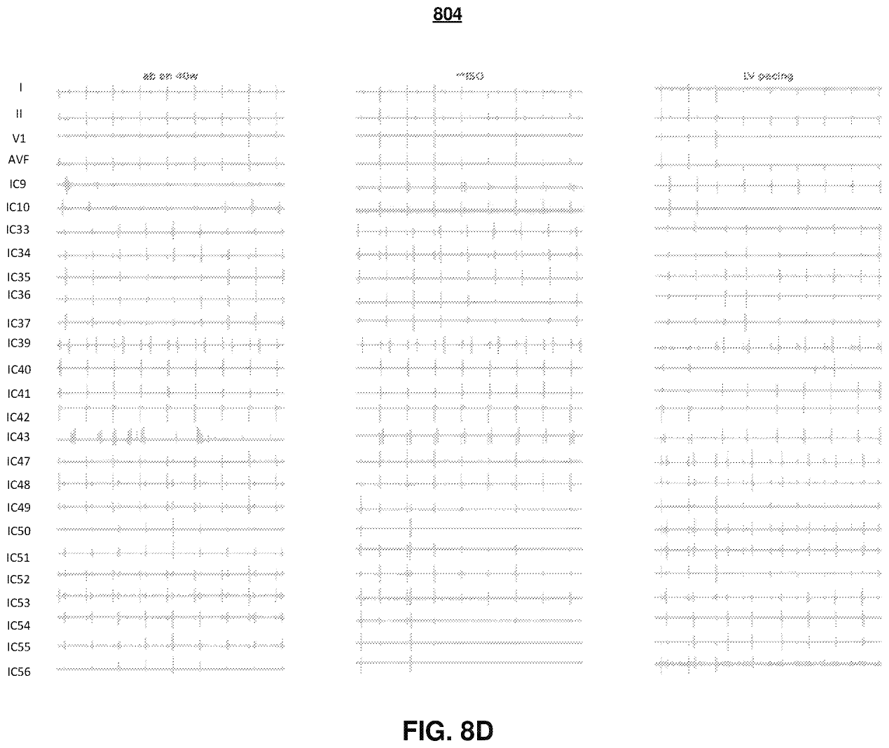

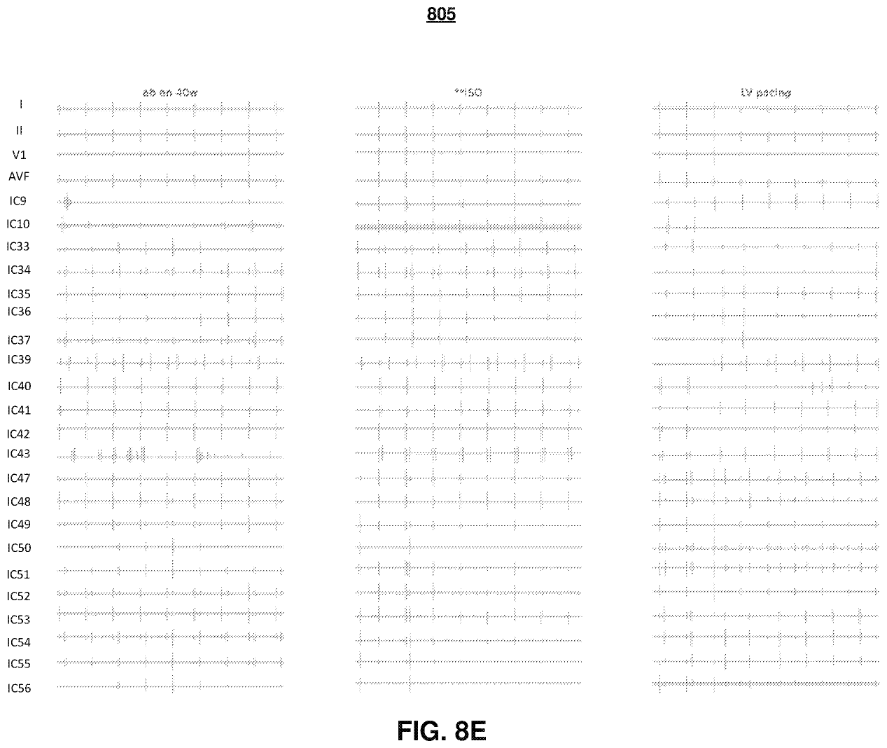

Referring to FIGS. 8A-8E, the plurality of EP signals after the detrending process as described in FIG. 2 are shown. In comparison, before the detrending process, the plurality of EP signals of the electrodes IC9, IC39, IC40, IC41, and IC43 disturbed by the injected energy before the detrending process as shown in FIG. 3, the intrinsic biological signals of interest are obscured by the extrinsic signals cause by the external energy. Referring to FIG. 8A, after the detrending process, the corresponding detrended EP signals IC9, IC39, IC40, IC41, and IC43 recover all intrinsic biological pulses of interest.

As described above, the smoothing timescale is tunable. The impact of changing the smoothing timescale is shown between FIGS. 8A-8E. FIG. 8A illustrates multiple detrended EP signals based on a cubic S-G smoothing with a size of the moving window of a number of 1001 samples of each interval. That is, the EP signals are sampled at about two kilohertz, and the size of the moving window is about 500 milliseconds. FIG. 8B illustrates multiple detrended EP signals based on a cubic S-G smoothing with a size of the moving window of a number of 401 samples of each interval. FIG. 8C illustrates multiple detrended EP signals based on a cubic S-G smoothing with a size of the moving window of a number of 101 samples of each interval. FIG. 8D illustrates multiple detrended EP signals based on a cubic S-G smoothing with a size of the moving window of a number of 51 samples of each interval. FIG. 8E illustrates multiple detrended EP signals based on a cubic S-G smoothing with a size of the moving window of a number of 25 samples of each interval. Comparing to the waveforms of the detrended EP signals, it can be found that when the S-G smoothing has a lower sample rate, which indicates a larger smoothing timescale, the less details of the intrinsic biological features can be recovered.

It is noted that, the disclosure hardware, software, and/or a combination thereof can allow a user to make a choice of the detrending process for each channel. For example, as shown in FIGS. 3 and 8A-8E, there are 26 channels of EP signals coming in. The clinicians can select whether to detrend a single channel, and can select a smoothing method (e.g., spline smoothing, S-G smoothing, etc.), and can select whether to trigger the detrending process based on an identification of a voltage change larger than a slew threshold. Further, the clinicians can also set the values of any suitable hyper parameters in the detrending process, such as the slew threshold, the temporal radius, the smoothing timescale, etc., individually on a per channel basis. In some embodiments, the values of hyper parameters in the detrending process can also automatically determined or adjusted by the system, such that the 26 waveforms can be automatically aligned with a same time delay corresponding to a maximum smoothing timescale.

Based on the teachings contained in this disclosure, it will be apparent to persons skilled in the relevant art how to make and use embodiments of this disclosure using data processing devices, computer systems, or computer architectures other than that shown in FIG. 1. In particular, embodiments can operate with software, hardware, and/or operating system implementations other than those described herein.

Accordingly, methods, systems, and media detrending bioelectric signals are provided to recover intrinsic bioelectric signals after an energy injection event. In the present disclosure, cardiac procedures are used as examples to describe the method. A person of ordinary skilled in the art would know that the disclosed method can be applied to general bioelectric waveforms at a human body. Since in general a human body is only capable of producing relatively small potential differences over a relatively long timescales, any artificial voltages that are generated to any parts of a human body, such as the central nervous system, the peripheral nervous system, or any other part having electrical waveforms, can be filtered by using the disclosed method.

One aspect of the present disclosure provides a system for detrending a bioelectric signal. The system comprises a processor configured to: receive the bioelectric signal; identify at least one breakpoint section corresponds to a rapid change of amplitude of the bioelectric signal; smooth an amplitude of the bioelectric signal after the at least one breakpoint section; and reconstruct the bioelectric signal based on the smoothed amplitude of the bioelectric signal; reset of the breakpoint section to remove extrinsic components caused by a non-biological factor from the bioelectric signal.

In some embodiments, the system further comprises an electrode configured to collect the bioelectric signal, wherein the bioelectric signal comprises a cardiac electrophysiology signal.

In some embodiments, the processor is further configured to determine a slew threshold indicating a minimum amplitude change for identifying an energy injection event caused by the non-biological factor.

In some embodiments, the processor is further configured to determine if the rapid change of amplitude of the bioelectric signal is larger than the slew threshold and a time point of the rapid change.

In some embodiments, the processor is further configured to identify the at least one breakpoint section based on the determined time point and a parametric temporal radius around the determined time point.

In some embodiments, the processor is further configured to adjust at least one of the slew threshold, the parametric temporal radius, and a smoothing timescale based on a user input.

In some embodiments, the processor is further configured to identify a plurality of breakpoint sections from the bioelectric signal during a period of time. Each of the plurality of breakpoint sections corresponds to one of a plurality of rapid changes of amplitude of the bioelectric signal during the period of time.

In some embodiments, the processor is further configured to: partition the bioelectric signal into a plurality of intervals based on the plurality of breakpoint sections; and independently smooth the amplitude of the bioelectric signal for each interval between adjacent breakpoint sections.

In some embodiments, the processor is further configured to apply a cubic spline smoothing process to smooth the amplitude of the bioelectric signal.

In some embodiments, the processor is further configured to apply a cubic Savitzky-Golay smoothing process to smooth the amplitude of the bioelectric signal.

In some embodiments, the processor is further configured to reset the amplitude of the bioelectric signal in the at least one breakpoint section to a ground baseline.

In some embodiments, the processor is further configured to subtract the smoothed amplitude of the bioelectric signal from the amplitude of the received bioelectric signal.

In some embodiments, the processor is further configured to: collect a plurality of bioelectric signals simultaneously; and simultaneously detrend at least two of the plurality of bioelectric signals in a real time basis.

Another aspect of the present disclosure provides a method for detrending a bioelectric signal. The method comprises: receiving the bioelectric signal; identifying at least one breakpoint section corresponds to a rapid change of amplitude of the bioelectric signal; smoothing an amplitude of the bioelectric signal after the at least one breakpoint section; and reconstructing the bioelectric signal based on the smoothed amplitude of the bioelectric signal and a reset of the breakpoint section to remove extrinsic components caused by a non-biological factor from the bioelectric signal.

In some embodiments, the method further comprises collecting a cardiac electrophysiology signal as the bioelectric signal.

In some embodiments, identifying the at least one breakpoint section comprises determining a slew threshold indicating a minimum amplitude change for identifying an energy injection event caused by the non-biological factor.

In some embodiments, identifying the at least one breakpoint section further comprises determining if the rapid change of the amplitude of the bioelectric signal is larger than the slew threshold and a time point of the rapid change.

In some embodiments, identifying the at least one breakpoint section further comprises identifying the at least one breakpoint section based on the determined time point and a parametric temporal radius around the determined time point.

In some embodiments, the method further comprises adjusting at least one of the slew threshold, the parametric temporal radius, and a smoothing timescale based on a user input.

In some embodiments, the method further comprises identifying a plurality of breakpoint sections from the bioelectric signal during a period of time. Each of the plurality of breakpoint sections corresponds to one of a plurality of rapid changes of amplitude of the bioelectric signal during the period of time.

In some embodiments, the method further comprises: partitioning the bioelectric signal into a plurality of intervals based on the plurality of breakpoint sections; and independently smoothing the amplitude of the bioelectric signal for each interval between adjacent breakpoint sections.

In some embodiments, smoothing the amplitude of the bioelectric signal comprises applying a cubic spline smoothing process to smooth the amplitude of the bioelectric signal.

In some embodiments, smoothing the amplitude of the bioelectric signal comprises applying a cubic Savitzky-Golay smoothing process to smooth the amplitude of the bioelectric signal.

In some embodiments, reconstructing the bioelectric signal comprises resetting the amplitude of the bioelectric signal in the at least one breakpoint section to a ground baseline.

In some embodiments, reconstructing the bioelectric signal further comprises subtracting the smoothed amplitude of the bioelectric signal from the original amplitude of the bioelectric signal.

In some embodiments, the method further comprises: collecting a plurality of bioelectric signals simultaneously; and simultaneously detrending at least two of the plurality of bioelectric signals in a real time basis.

Another aspect of the present disclosure provides a non-transitory computer-readable medium containing computer-executable instructions that, when executed by a hardware processor, cause the hardware processor to perform a method for detrending bioelectric signals, as described above.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible and are within the scope and spirit of this disclosure. For example, and without limiting the generality of this paragraph, embodiments are not limited to the software, hardware, firmware, or entities illustrated in the figures or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein. This disclosure also extends to methods associated with using or otherwise implementing the features of the disclosed hardware and systems herein.

The breadth and scope of this disclosure should not be limited by any of the above-described exemplary embodiments, but should be defined only in accordance with the following claims and their equivalents.

What is claimed is:

1. A system for detrending a bioelectric signal, comprising:
a processor configured to:
receive the bioelectric signal;
identify at least one breakpoint section corresponds to a rapid change of amplitude of the bioelectric signal;
smooth an amplitude of the bioelectric signal after the at least one breakpoint section; and
reconstruct the bioelectric signal based on the smoothed amplitude of the bioelectric signal and a reset of the breakpoint section to remove extrinsic components caused by a non-biological factor from the bioelectric signal.

2. The system of claim 1, further comprising:
an electrode configured to collect the bioelectric signal, wherein the bioelectric signal comprises a cardiac electrophysiology signal.

3. The system of claim 1, wherein the processor is further configured to:
determine a slew threshold indicating a minimum amplitude change for identifying an energy injection event caused by the non-biological factor;
determine if the rapid change of amplitude of the bioelectric signal is larger than the slew threshold and a time point of the rapid change; and
identify the at least one breakpoint section based on the determined time point and a parametric temporal radius around the determined time point.

4. The system of claim 3, wherein the processor is further configured to:
adjust at least one of the slew threshold, the parametric temporal radius, and a smoothing timescale based on a user input.

5. The system of claim 1, wherein the processor is further configured to:
identify a plurality of breakpoint sections from the bioelectric signal during a period of time,
wherein each of the plurality of breakpoint sections corresponds to one of a plurality of rapid changes of amplitude of the bioelectric signal during the period of time.

6. The system of claim 5, wherein the processor is further configured to:
partition the bioelectric signal into a plurality of intervals based on the plurality of breakpoint sections; and
independently smooth the amplitude of the bioelectric signal for each interval between adjacent breakpoint sections.

7. The system of claim 1, wherein the processor is further configured to:
apply a cubic spline smoothing process or a cubic Savitzky-Golay smoothing process to smooth the amplitude of the bioelectric signal.

8. The system of claim 1, wherein the processor is further configured to:
reset the amplitude of the bioelectric signal in the at least one breakpoint section to a ground baseline.

9. The system of claim 1, wherein the processor is further configured to:
subtract the smoothed amplitude of the bioelectric signal from the amplitude of the received bioelectric signal.

10. The system of claim 1, wherein the processor is further configured to:
collect a plurality of bioelectric signals simultaneously; and
simultaneously detrend at least two of the plurality of bioelectric signals in a real time basis.

11. A method for detrending a bioelectric signal, comprising:
receiving the bioelectric signal;
identifying at least one breakpoint section corresponds to a rapid change of amplitude of the bioelectric signal;
smoothing an amplitude of the bioelectric signal after the at least one breakpoint section; and
reconstructing the bioelectric signal based on the smoothed amplitude of the bioelectric signal and a reset of the breakpoint section to remove extrinsic components caused by a non-biological factor from the bioelectric signal.

12. The method of claim 11, further comprising:
collecting a cardiac electrophysiology signal as the bioelectric signal.

13. The method of claim 11, wherein identifying the at least one breakpoint section comprises:
determining a slew threshold indicating a minimum amplitude change for identifying an energy injection event caused by the non-biological factor;
determining if the rapid change of the amplitude of the bioelectric signal is larger than the slew threshold and a time point of the rapid change; and
identifying the at least one breakpoint section based on the determined time point and a parametric temporal radius around the determined time point.

14. The method of claim 13, further comprising:
adjusting at least one of the slew threshold, the parametric temporal radius, and a smoothing timescale based on a user input.

15. The method of claim 14, further comprising:
identifying a plurality of breakpoint sections from the bioelectric signal during a period of time, wherein each of the plurality of breakpoint sections corresponds to one of a plurality of rapid changes of amplitude of the bioelectric signal during the period of time.

16. The method of claim 15, further comprising:
partitioning the bioelectric signal into a plurality of intervals based on the plurality of breakpoint sections; and
independently smoothing the amplitude of the bioelectric signal for each interval between adjacent breakpoint sections.

17. The method of claim 11, wherein smoothing the amplitude of the bioelectric signal comprises:
applying a cubic spline smoothing process or a cubic Savitzky-Golay smoothing process to smooth the amplitude of the bioelectric signal.

18. The method of claim 11, wherein reconstructing the bioelectric signal comprises:
resetting the amplitude of the bioelectric signal in the at least one breakpoint section to a ground baseline.

19. The method of claim 11, wherein reconstructing the bioelectric signal comprises:
subtracting the smoothed amplitude of the bioelectric signal from the original amplitude of the bioelectric signal.

20. The method of claim 11, further comprising:
collecting a plurality of bioelectric signals simultaneously; and
simultaneously detrending at least two of the plurality of bioelectric signals in a real time basis.
